(19) United States
(12) Patent Application Publication
(10) Pub. No.: US 2005/0266039 A1
(43) Pub. Date: Dec. 1, 2005

(54) Coated medical device and method for making the same

(76) Inventor: Jan Weber, Maple Grove, MN (US)

Correspondence Address: JONES DAY, 222 EAST 41ST ST, NEW YORK, NY, 10017, US

(21) Appl. No.: 10/857,723

(22) Filed: May 27, 2004

(51) Int. Cl. A61K 31/337; A61F 2/00

(52) U.S. Cl. 424/423; 514/449

(57) ABSTRACT

The invention pertains to medical devices, such as stents, having a surface and a first coating layer comprising a first polymer disposed on at least a portion of the surface, in which at least one cavity formed in the first coating layer. A biologically active material is deposited into the cavity, and a second coating layer comprising a second polymer is disposed over the biologically active material in the cavity. The cavity may be formed using an excimer laser or ultrashort laser to ablate the first coating layer, and the biologically active material may be deposited in the cavity using a picoliter dispensing system. Methods for making such medical devices are also disclosed.

### FIELD OF THE INVENTION

This invention relates generally to medical devices for delivering a biologically active material to a desired location within the body of a patient. More particularly, the invention is directed to medical devices having a first coating layer disposed on at least a portion of the surface of the medical device with at least one cavity in the first coating layer. A biologically active material is deposited in the cavity and a second coating layer is disposed over the biologically active material in the cavity. The invention is also directed to a method for manufacturing a coated medical device.

### BACKGROUND OF THE INVENTION

It has been common to treat a variety of medical conditions by introducing an insertable or implantable medical device having a coating for release of a biologically active material. For example, various types of drug-coated stents have been used for localized delivery of drugs to a body lumen. See, e.g., U.S. Pat. No. 6,099,562 to Ding et al.

However, there can be difficulties associated with the manufacture and delivery of a medical device having a coating that includes a precise amount of drug or drugs. Generally, such medical devices are manufactured by coating the surface of a medical device with a drug. To apply the drug to the surface of the medical device, the drug may be pre-mixed with a polymer and then applied to the surface of a medical device.

A common technique used to apply a drug mixture to a medical device is by spray-coating. To use this method, the drug generally must be well dispersed through a polymer coating formulation. But there are some difficulties associated with using a spray-coating method to apply a drug coating on a medical device. For instance, it is often difficult to disperse the drug or biologically active material of choice in a polymer coating mixture or formulation. Moreover, it may not be possible to dissolve the drug in the same solution as the polymer.

Also, because the drug or biologically active material can only tolerate a certain range of temperatures, the temperature at which the coating is dried or cured is restricted by the presence of the drug or biologically active material in the coating. More specifically, if the drug or biologically active material has a maximum temperature tolerance of 50° C., the polymer coating containing such drug or material should not be dried or cured above this temperature or the drug or biologically active material may lose its efficacy. Therefore, an application of a coating formulation that contains both a drug or biologically active material and polymer to a medical device can limit the temperature at which the coating is dried or cured and increase the amount of drying time required.

In addition, it is often desirable to have a medical device that is coating with two or more drugs that are not mixed together. With conventional technologies it is difficult to create a medical device having more than one drug wherein the drugs are not mixed.

It is also difficult to create a stent having a release profile that is different at the ends of the stent than the release profile of the drug in the middle of the stent.

A further limitation of the present methods for applying a coating to a medical device is the inability to position the biologically active material only in predefined regions on the medical device, such as only on the distal and proximal ends of a stent. More specifically, in the conventional methods for coating medical devices, such as spray-coating or dipping, an entire surface or all surfaces of the medical device are coated even though it may be desired that only part of the surface is coated, or only some of the surfaces are coated. For instance, in medical devices having a tubular portion, such as a vascular stent, the inner surface of the tubular portion does not need to be coated with a coating containing a biologically active material that is used to treat only the body lumen wall that contacts the outer surface of the stent. This is because the inner surface of the stent does not come in contact with a body-lumen wall and does not apply the biologically active material to the body-lumen wall. When all the surfaces of a medical device such as a stent, including surfaces that are not directly in contact with the body tissue of a patient, are coated with a composition comprising a biologically active material, more biologically active material is used than is needed. Thus, the patient may receive unnecessary exposure to the material. Likewise, when the entire outer surface of a medical device contains a biologically active material, this biologically active material can be delivered to both tissues in need of treatment, such as lesions and healthy body tissue. Treatment of healthy tissue with the biologically active material is not only unnecessary but maybe harmful. Also, manufacturing costs for the medical device may needlessly increase by including unnecessary amounts of the biologically active material in the medical device.

Also, with existing coated medical devices, generally, the coating is uniformly applied along the entire length of the device or surface of the device. For example, conventional coated stents are coated uniformly along the entire length of their surface. By having the device uniformly coated along its length, the concentration release profile of the biologically active material along the length of the coated surface may be in the shape of a bell-curve, wherein the concentration of the biologically active material released at the middle of the surface is greater than the concentration of the biologically active material released at the ends of the coated surface. This concentration-release profile may lead to the delivery of an inadequate or sub-optimal dosage of the biologically active material to the body tissue located in the proximity of the ends of the coated medical device. It is possible that such insufficient delivery of the biologically active material may lead to undesired effects. For example, in the case of a biologically active material-coated stent used to prevent or treat restenosis, if the amount of biologically active material delivered to the tissue located at the ends of the stent is sub-optimal, it is possible that restenosis may occur in such tissue. Accordingly, there is a need for coated medical devices where the biologically active material can be positioned in predefined or selected regions of the medical device.

Another disadvantage of conventional coating methods is its low efficiency resulting from the fact that only a small percentage of the coating material applied to the medical device adheres to the medical device. For instance, in spray-coating methods, between 30 to 95% of the coating composition may be lost. Such inefficiency can be very costly, particularly when applying expensive drugs such as DNA or viruses. Thus, there is also a need for an efficient and cost-effective method of manufacturing coated medical device.

### SUMMARY OF THE INVENTION

These and other objectives are accomplished by the present invention. To achieve the aforementioned objectives, we have invented a coated medical device, such as a stent, comprising: a medical device having a surface; a first coating layer comprising a first polymer disposed on at least a portion of the surface; and at least one cavity in the first coating layer. A first biologically active material is deposited into the cavity, and a second coating layer comprising a second polymer is disposed over the first biologically active material in the cavity.

The present invention also provides for a stent that has a sidewall comprising a plurality of struts having a surface. In this embodiment, a first coating layer comprising a polymer is disposed on at least a portion of the surface of at least one strut; and a plurality of cavities are formed in the first coating layer by ablation with a laser. A biologically active material is deposited in the cavities; and a second coating layer comprising a polymer is disposed over the biologically active material in the cavities.

In another embodiment, a first coating layer comprising a polymer is disposed on at least a portion of the surface of a medical device; and at least one cavity is formed in the first coating layer such as by ablation with a laser. A second polymer mixed with a biologically active material is deposited in the cavity to form a second coating layer.

Also described herein is a method for manufacturing such a medical device. This method comprises forming on the surface of a medical device a first coating layer comprising a first polymer; and laser ablating at least one cavity in the first coating layer. The method further comprises depositing a biologically active material in at least a portion of the cavity; and forming a second coating layer comprising a second polymer over the biologically active material in the cavity. The cavity may be formed using an excimer laser to ablate the first coating layer, and the biologically active material may be deposited in the cavity using a picoliter dispensing system.

The present invention provides for a coated medical device in which amounts of biologically active material can be precisely located or positioned on the medical device. Also, the present invention provides for an efficient and effective method of manufacturing a medical device by depositing a precise amount of a biologically active material onto the medical device, with little loss of the biologically active material during the coating of the medical device. Thus, a desired release profile may be created and one or more drugs may be accurately positioned on a medical device.

### BRIEF DESCRIPTION OF THE DRAWINGS

FIGS. 1a and 1b are cross-sectional views of a portion of a medical device of the present invention showing a medical device having a surface, a first coating layer on the surface, a cavity in the first coating layer, a biologically active material in the cavity and a second coating layer disposed over the biologically active material.

FIGS. 2a and 2b are cross-sectional views of other embodiments of a medical device of the present invention showing a medical device, a surface of the medical device, a first coating layer, a cavity, a biologically active material in the cavity and a second coating layer disposed over the biologically active material and the first coating layer.

FIG. 3 is a perspective view of a stent having a first coating layer and cavities therein.

FIG. 4 is a cross-sectional view of an embodiment of the present invention in which the cavities in the first coating layer are of varying sizes or volumes.

FIG. 5 is a schematic diagram showing a method of manufacturing a medical device of the present invention.

### DETAILED DESCRIPTION OF THE INVENTION

The medical devices of the present invention can be inserted into and implanted in the body of a patient. The medical devices suitable for the present invention include, but are not limited to, stents, surgical staples, catheters, such as central venous catheters and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, hemodialysis units, hemoperfusion units and plasmapheresis units.

Medical devices suitable for the present invention include those that have any shape, such as a tubular or cylindrical-like portion, as long as the medical device or subassemblies of the medical device are accessible by laser. The tubular portion of the medical device need not be completely cylindrical. For instance, the cross-section of the tubular portion can be any shape, such as rectangle, a triangle, etc., not just a circle. Such devices include, without limitation, stents and grafts. A bifurcated stent is also included among the medical devices which can be fabricated by the method of the present invention. In addition, the tubular portion of the medical device may be a sidewall that is comprised of a plurality of struts. The struts may be arranged in any suitable configuration. Also, the struts do not all have to have the same shape or geometric configuration. Each individual strut has a surface adapted for exposure to the body tissue of the patient. The tubular sidewall may be a stent.

Medical devices that are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

The medical devices suitable for the present invention may be fabricated from metallic, ceramic, or polymeric materials, or a combination thereof. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Suitable ceramic materials include, but are not limited to, titaniumoxides, iridiumoxides, and hafnium oxides.

Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

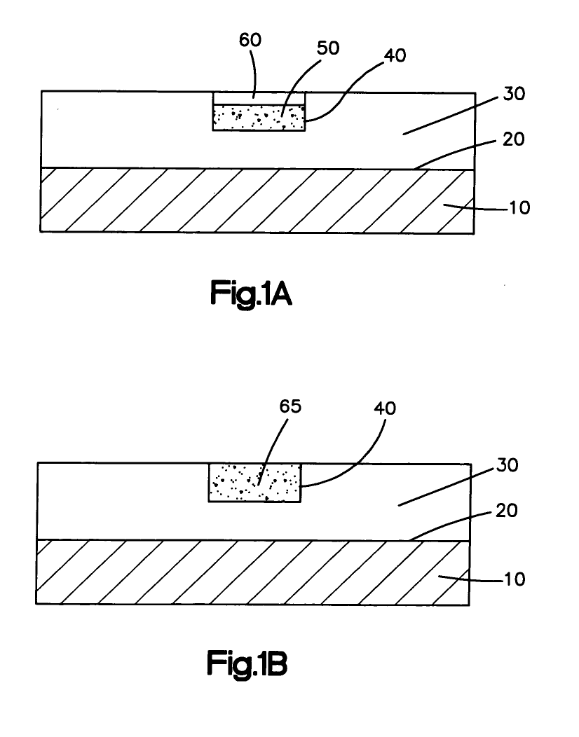

As shown in FIGS. 1a-b and 2a-b, in embodiments of the present invention, the insertable or implantable portion of the medical device 10 of the present invention has a surface 20. When the medical device 10 is a stent comprising a plurality of struts, the surface 20 is located on a strut. When the medical device 10 is a stent covered by a graft material, for example, ePTFE or polyester, the surface is located on the graft material. The cavities may therefore be formed in a coating disposed on the surface of the graft material or in the surface of the graft material.

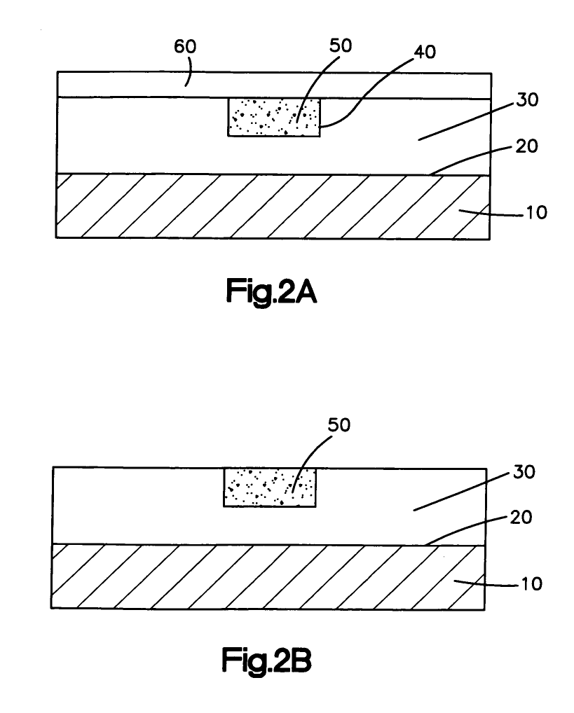

A first coating layer 30 is disposed over the surface 20 of the medical device 10, as shown in FIGS. 1a-b and 2a-b. FIG. 1a is a cross-sectional view of a portion of a medical device 10 having a surface 20, and a first coating layer 30 on the surface 20. A cavity 40 is formed in the first coating layer. The cavity 40 contains a biologically active material 50. A second coating layer 60 comprising a second polymer is disposed over the biologically active material 50. FIG. 2a is a cross-sectional view of another embodiment of a medical device 10 of the present invention showing a portion of a medical device 10 having a surface 20, and a first coating layer 30 disposed over the surface 20. A cavity 40 is disposed in the first coating layer 30. A biologically active material 50 is contained in the cavity 40. Unlike the embodiment in FIG. 1a, the second coating layer 60, not only covers the biologically active material 50 but also the first coating layer 30.

In another embodiment, the biologically active material is mixed with the second coating layer polymer and then the mixture is deposited in the cavity 40. FIG. 1b is a cross-sectional view of a portion of a medical device 10 having a surface 20, and a first coating layer 30 on the surface 20. A cavity 40 is disposed in the first coating layer. The cavity 40 contains a second coating layer 65 that comprises a biologically active material and a second polymer.

FIG. 2b shows a cross-sectional view of a portion of a medical device in which the biologically active material 50 is not covered by a second coating layer 60. In such case, the biologically active material 50 and a solvent that is compatible with the first coating layer 30 are injected into the cavity 40 on the first coating layer 30 so that the polymer of the first coating layer 30 mixes with the biologically active material 50. In one embodiment, the biologically active material and a polymer are dissolved in a solvent to form a solution. The solution is then injected into the cavity. In another embodiment, a solvent is injected into the cavity. The solvent is allowed to dissolve the surface of the cavity, after which, the biologically active material and a polymer is added. The cavity may or may not be filled entirely. In a specific embodiment, the cavity is only filled at the bottom. In such a case, any sheering force that is applied to the coating during delivery of the device to the body lumen will not affect the total amount of drug in the cavity.

The first coating layer 30 is preferably formed by applying a first coating composition to the surface 20. Coating compositions suitable for applying to the devices of the present invention include a polymeric material dispersed or dissolved in a solvent suitable for the medical device 10, which are known to the skilled artisan. Preferably, the first coating layer is substantially free of a biologically active material. By having the first coating layer substantially free of a biologically active material, a biologically active material can be more effectively positioned in selected locations on the surface of the medical device.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material are styrene-isobutylene-styrene copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing. Suitable polymers also include bioabsorbable polymers such as, but not limited to, poly(DL-(lactic-co-glycolic acid) (PLGA) and poly(L-lactic acid) (PLLA).

More preferably for medical devices which undergo mechanical challenges, e.g., expansion and contraction, the polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

The solvents used to prepare coating compositions include ones which can dissolve the polymeric material into solution or suspend the polymeric material. Examples of suitable solvents include, but are not limited to, tetrahydrofuran, methylethylketone, chloroform, toluene, acetone, isooctane, 1,1,1,-trichloroethane, dichloromethane, isopropanol, and mixture thereof.

The first coating composition can be applied by any method to a surface 20 of a medical device 10 to form a coating layer. Examples of suitable methods include, but are not limited to, spraying such as by conventional nozzle or ultrasonic nozzle, dipping, rolling, electrostatic deposition, and a batch process such as air suspension, pancoating or ultrasonic mist spraying. Also, more than one coating method can be used to make a medical device 10.

The first coating layer 30 has at least one cavity 40 formed therein as shown in FIGS. 1 and 2. Preferably, the first coating layer 30 has a plurality of cavities 40 formed therein. The term “cavity” refers to an indentation, receptacle or groove of any cross-sectional configuration, depth, shape, volume, width or size. The cavities 40 can be situated in a regular pattern, such as in a row, or in an irregular manner. The cavities 40 may be spaced apart any desired distance. Preferably, the cavities 40 do not overlap.

Also, the cavities 40 can be but do not have to be disposed evenly on the entire surface 20 of the medical device 10. Any surface density of the cavities 40 may be created. For example, the surface density of the cavities may be uniform along the circumference of the medical device. Also, the cavities 40 may be localized in one or more areas on the surface 20 of the medical device 10 while other areas of the device do not have cavities 40 in the first coating layer 30. For example, the cavities 40 may be more densely disposed on the surface 20 in areas where a stronger release of the biologically active material is desired. With a stent, the surface density may be greater at the end of the stent to have an additional effect of the release of the biologically active material outside of the stent. In particular, there may be a higher concentration of cavities such that there is a greater amount of biologically active material at the proximal end of a stent, or both ends of the stent may have a higher concentration of cavities. In addition, to obtain a uniform distribution of the biologically active material, it may be necessary to have a nonuniform distribution of the cavities. For instance, with a stent having struts of varying thickness, it may be desirable to have more cavities on the thinner struts.

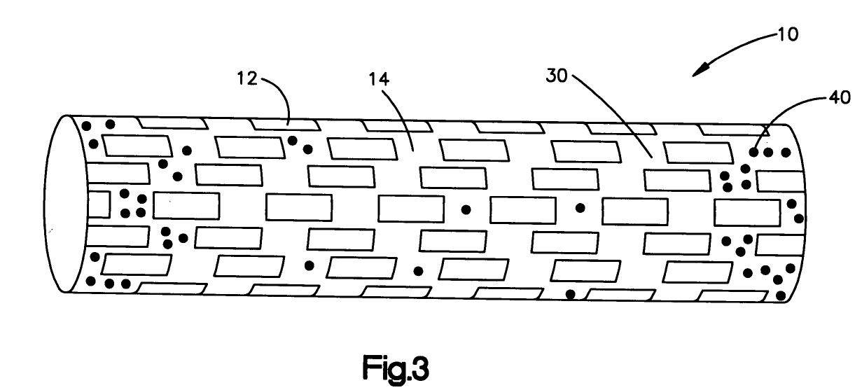

FIG. 3 shows a perspective view of a stent or medical device 10 having a first coating layer 30 wherein the surface density of cavities 40 in the first coating layer 30 is greater at the ends of the stent than in the middle of the medical device 10. The medical device 10 is comprised of a plurality of struts 14 and openings 12. A first coating layer 30 is disposed over the surface of the strut 14. A plurality of cavities 40 are formed in the first coating layer 30. However, the surface density of the cavities 40 is greater at the ends of the medical device 10 than in the middle of the medical device 10.

The cavities 40 may have the same depth or volume. On the other hand, the depth or volume of the cavities may vary from cavity to cavity. A cavity 40 may extend to the surface 20 of the medical device 10 such that the depth of the cavity 40 is equal to the thickness of the first coating layer 30. Preferably, the depth of the cavity 40 is less than the thickness of the first coating layer 30. It may be desirable to apply a thicker first coating layer 30 so that the cavities 40 may have greater depths or can have more varied depths. Deeper cavities 40 or cavities having greater volume may be created at the end of a stent to accommodate larger amounts of the biologically active material and allow for more biologically active material to be released from the ends as compared to the release of the biologically active material from the cavities 40 in the middle section of the stent.

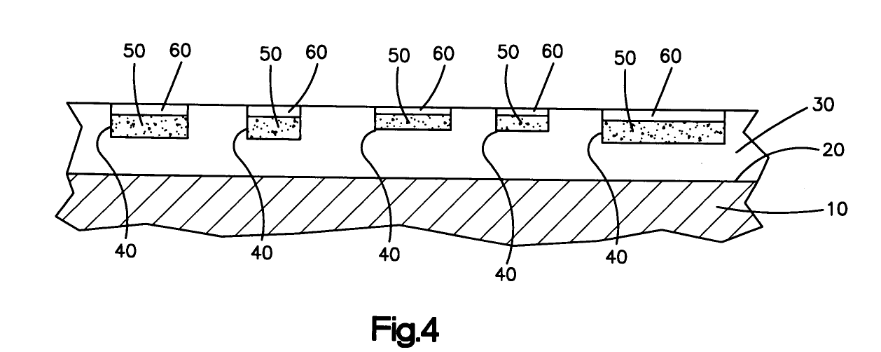

FIG. 4 shows a cross-sectional view of a medical device 10 having a first coating layer 30 disposed over the surface 20 of the medical device. The first coating layer 30 comprises a plurality of cavities 40. The cavities have varying depths and/or volumes for containing varying amounts of a biologically active material 50.

The cavities 40 in the first coating layer 30 can be formed by chemical etching, photo-etching, high-velocity particle impact (“blast methods”), stamping or laser ablation such as by an excimer laser, a YAK laser, or an ultrashort laser. See, e.g., U.S. Pat. No. 6,517,888 B1. Preferably, the cavities 40 are formed by ablation of the first coating layer 30 using an excimer laser.

The cavities 40 may be formed by ablation of the first coating layer 30 using an excimer laser with any wavelenth. Excimer lasers operate with wavelengths in the ultraviolet region, such as 157, 193, 248, 308 or 351 nm, depending on the gas mixture used. By varying the gas mixture of the excimer laser, the wavelength may be varied to adjust the ablation as know to one skilled in the art.

The short wavelengths of excimer lasers are strongly absorbed in most materials including polymers. Ablation will only take place at energy densities above a threshold which is different for all materials. The threshhold is significantly lower for polymers as compared to ceramic materials and metals. Therefore it is possible, in certain combinations of materials, that a selective process can take place. In addition, the first coating layer may be ablated without affecting the medical device.

Very small cavities 40 to the order of a few micrometers can be produced with ultrashort lasers or the short wavelengths of the excimer laser. The size of the ablated cavity 40 can be as small as about 3 micrometer or less, but can be made larger in size. Different sized cavities 40 may be created by changing the focus of the laser beam as known to one skilled in the art. Preferably, different masks are used to create cavities having different shapes. For example, using a mask with small holes or projecting a mask, by optical means, with small holes can be used to ablate small holes in the first coating layer.

The excimer laser is a pulsed laser with extremely short pulse durations of about 20 nanoseconds. The pulse delivers an energy of a few hundred millijoules. Thus, during a pulse a power of 10-30 MWatt is present. The repetition rate may be up to a few hundred Hertz, so that the average output power is between about 50 and 150 Watts.

The penetration depth of an excimer laser is about 0.1 to about 2 μm, so that all energy will be absorbed in a very thin first coating layer 30. Each pulse of the laser will ablate a certain volume. The ablation depth is between 0.1-10 μm for a single pulse, depending on the amount and the adsorption of the energy in the polymer. To ablate a larger volume, more pulses are required. Also, the higher the repetition rate of the pulse, the shorter the total time required to ablate the cavities 40 in the first polymer layer. Thus, ablation using an excimer laser is very accurate because a cavity 40 of a defined depth may be created by applying a certain number of pulses.

Because an excimer laser has a very short pulse time and a high peak power, ablation of the first coating layer 30 to form a cavity 40 will not damage the portion of the first coating layer 30 that surrounds the cavity 40. There is very low to no thermal influence to the area outside the cavity 40 when using an excimer laser or ultrashort laser.

Generally, the cavities 40 do not extend to the medical device 10 itself so that the laser does not affect the material of the medical device 10. However, if it is desired to create cavities 40 that extend to the surface 20 of the medical device 10, the medical device 10 should be made of a material that is not affected by the excimer laser.

After formation of a cavity 40 in the first coating layer 30, a biologically active material 50 is deposited into the cavity 40 as shown in FIGS. 1, 2 and 4. The biologically active material 50 may partially or completely fill the cavity 40.

The term “biologically active material” encompasses therapeutic agents, such as drugs, and also genetic materials and biological materials. The genetic materials mean DNA or RNA, including, without limitation, of DNA/RNA encoding a useful protein stated below, intended to be inserted into a human body including viral vectors and non-viral vectors as well as anti-sense nucleic acid molecules such as DNA, RNA and RNAi. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPPATEK), lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endothelial Mitogenic Growth Factors, and epidermal growth factors, transforming growth factor and platelet derived endothelial growth factor, platelet derived growth factor, tumor necrosis factor, hepatocyte growth factor and insulin like growth factor), transcription factors, proteinkinases, CD inhibitors, thymidine kinase, monoclonal antibodies, and bone morphogenic proteins (BMP's), such as BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material 50 also includes non-genetic therapeutic agents, such as:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promotors such as growth factors, Vascular Endothelial Growth Factors (FEGF, all types including VEGF-2), growth factor receptors, transcriptional activators, and translational promotors;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin; rapamycin (sirolimus);
angiogenic substances, such as acidic and basic fibrobrast growth factors, estrogen including estradiol (E2), estriol (E3) and 17-Beta Estradiol;
drugs for heart failure, such as digoxin, beta-blockers, angiotensin-converting enzyme (ACE) inhibitors including captopril and enalopril, statins and related compounds;
fat-soluble vitamins A, D, E, K and their derivatives;
cortisone and its derivatives; and
immunosuppressant, such as sirolimus or rapamycin.

Preferred biologically active materials include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred biologically active materials include nitroglycerin, nitrous oxides, nitric oxides, antibiotics, aspirins, digitalis, estrogen derivatives such as estradiol and glycosides.

The biologically active material 50 may be applied alone or with other materials such as a solvent. Suitable solvents include, but are not limited to, tetrahydrofuran, methylethylketone, chloroform, toluene, acetone, isooctane, 1,1,1,-trichloroethane, dichloromethane, isopropanol, water and mixture thereof. The solvent may be mixed with the biologically active material 50 before being deposited in the cavity 40 or the biologically active material 50 may be deposited first and then the solvent applied over the biologically active material 50. In an embodiment, biologically active material such as genetic material that is dissolved in water may be frozen and used in a blast method to form cavities in the coating layer. The water may be evaporated from the cavities, leaving the biologically active material in the cavities.

Furthermore, the biologically active material 50 may also be applied with a polymer. Suitable polymers include, but are not limited to, those listed above with respect to the first coating layer 30. Moreover, bioabsorbable polymers can be used. Suitable bioabsorbable polymers include, but are not limited to, polysacharides, PVA and PLLA. In other embodiments, the topcoating may be made of “bucky paper,” which is a highly biocompatible layer of single wall carbon nano tubes.

The biologically active material 50 may be deposited into the cavity 40 using any suitable method as known to one skilled in the art. Preferably, a picoliter dispensing system using piezo technology is used to fill the cavities 40 with the biologically active material 50. Picoliter dispensing systems are commercially available. For example, Microdrop manufactures such a dispensing system. A picoliter dispensing system allows for the precise measurement of the amount of biologically active material 50 dispensed and can also be automated and computer-controlled for efficient manufacture of the medical device 10. A picoliter dispensing system deposits one droplet of the biologically active material 50 at a time, thus allowing for precise measurement. Laser scanning may be used to determine the precise size of each droplet of the biologically active material 50 that is deposited in a cavity 40. For example, a PDPA (phase dopler particle analyzer) method could be used. Another method would be to use a stroboscope, in conjunction with a camera, to measure the diameter of each droplet of the biologically active material. A single drop of the biologically active material 50 can be as small as 30 picoliter which results in ball-like droplets with a diameter of about 30 micrometer. Preferably, droplets are smaller than the diameter of the struts. The cavity 40 need not be completely filled with the biologically active material 50 and can only be partially filled. Also, the plurality of cavities 40 in the first coating layer 30 can be filled with varying amounts of biologically active materials 50.

After the biologically active material 50 has been deposited in the cavity 40, a second coating layer 60 (See FIGS. 1, 2 and 4) may be disposed over the biologically active material 50 in the cavity 40. The second coating layer 60 preferably includes a second polymer. The second polymer may be the same as or different than the first polymer in the first coating layer 30. The second coating layer 60 preferably should be able to form a bond with the first coating layer 30. Preferably, the second polymer is the same as the first polymer, or at least be from the same family as the first polymer. The second layer may also be “bucky paper”, a highly biocompatible layer of single wall carbon nano tubes.

In addition, the second polymer may be fashioned to create a desired release profile of the biologically active material 50, such as by adjusting the thickness of the second coating layer 60. Also, the thickness of the second coating layer 60 that is disposed over the biologically active material may vary from cavity to cavity 40. Another method for creating a desired release profile is to deposit a mixture of a biologically active and a polymer into the ablated cavity.

The second coating layer 60 may also include other materials such as a biologically active material 50 which may be the same as or different than the biologically active material 50 deposited into the cavity 40 and onto which the second coating layer 60 is applied. On the other hand, the second coating layer 60 can be substantially free of any biologically active material 50. If the biologically active material 50 is first mixed with a polymer before being deposited in a cavity 40, a polymeric second coating layer 50 may not be necessary.

The second coating layer 60 may be of any thickness. In addition, the second coating layer 60 may fill the cavity 40 to cover the biologically active material 50 or cover the biologically active material 50 and also at least a portion of the first coating layer 30. As shown in FIGS. 1 and 4, the second coating layer 60 covers the biologically active material 50 and fills the cavity 40 and is level with the first coating layer 30. In FIG. 2, the second layer covers the biologically active material 50 and the first coating layer 30.

The second coating layer 60 is preferably formed by applying a second coating composition over the biologically active material 50. The coating composition may also be applied over at least a portion of the first coating layer 30. The second coating composition includes a polymer. Preferably, the polymer is dispersed or dissolved in a solvent. Any of the polymers and solvents listed above with respect to the first coating composition may be used to prepare the second coating composition.

The second coating layer 60 may be applied using any suitable method as known to one skilled in the art. For example, another picoliter dispensing system may be used to cover the biologically active material 50 with a second coating composition comprising a second polymer disposed over the biologically active material 50 in the cavity 40.

By adjusting the depth and size of the cavities 40, the amount of biologically active material 50 and the thickness of the first and second coating layers 30, 60, a desired release profile may be achieved for the biologically active material 50. The biologically active material 50 travels e.g., by diffusion or elution, through the second coating layer 60 to the body lumen. Thus, creating cavities 40 in which the biologically active material 50 is deposited allows for a controlled release of the biologically active material 50. For example, a thicker first coating layer 30 with cavities 40 having different depths may provide a more sustained release profile as it would be more difficult for the biologically active material 50 to diffuse through the first coating layer 30.

In addition, by creating cavities 40 at desired locations on the surface 20 of the medical device 10 and filling the cavities 40 with a certain amounts of a biologically active material 50 results in a cost savings because unnecessary amounts of such biologically active material 50 are not applied to locations on the surface 20 where the biologically active material 50 should not be present. Also, the patient is not exposed to unnecessary dosages of such biologically active materials 50.

Furthermore, because the polymer and drug or biologically active material can be separately applied to the medical device 60 to form a coating containing both the polymers and drug or biologically active material, the latter does not have to be dispersed in a polymer mixture before being applied to the medical device. Also, because the drug or biologically active material is not combined in a coating formulation with the polymer, a coating formulation containing the polymer can be dried or cured at a temperature that is not limited by the temperature tolerance of the drug or biologically active material.

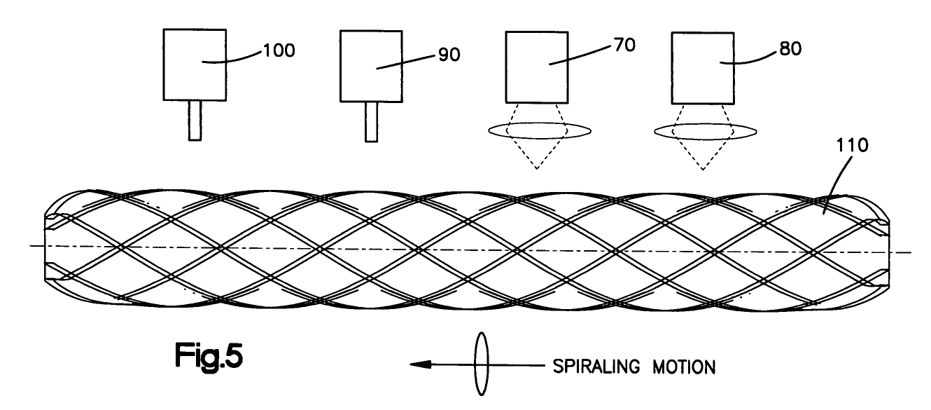

The present invention also comprises a method for manufacturing the medical device 10 described above. FIG. 5 shows a schematic drawing of a system used in the manufacturing of a medical device 10 of the present invention. As shown in FIG. 5, the medical device 10, which is mounted on a catheter 110, preferably moves in an axial direction in a spiraling motion. The system shown includes an excimer laser 70, a sensor 80, a first dispenser 90 for dispensing the biologically active material 50, and a second dispenser 100 for dispensing the second coating composition, all of which are fixed along the axial direction of the spiraling path of the medical device 10. In addition, the system is preferably automated. The method of the present invention and the systems are explained in greater detail below.

The method of the present invention comprises forming a first coating layer 30 on the surface 20 of a medical device 10 as discussed above. If the medical device 10 is a stent having a sidewall comprising a plurality of struts, the surface 20 is part of the struts.

The method further comprises ablating at least one cavity 40 in the first coating layer 30. A plurality of cavities 40 may be ablated in the first coating layer 30 and the cavities 40 may have different depths and/or volume. With a stent comprising a plurality of struts, the cavities 40 may be formed in any pattern on the outer surface of the struts and on the inner surface of the struts.

As described above, the cavity 40 is preferably formed using an excimer laser 70 to ablate the first coating layer 30. The ablation by the excimer laser 70 may be done by any suitable method. For example, when it is desired to ablate a plurality of cavities 40, the medical device 10 may be rotated and moved axially in relation to the excimer laser 70 as the excimer laser 70 ablates cavities 40 in the first coating layer 30 of the medical device 10 at fixed time intervals. For example, after a stent is coated with the first coating layer 30, the stent may be crimped on a balloon catheter and placed on a mandrel which allows the balloon and the stent to be rotated while moving axially in a spiral motion. Preferably, a fixed ratio of rotating speed to forward movement is used. The ratio between the rotation speed of a medical device 10, such as a stent, and the axial movement speed depends on the pattern of the stent. For example, if the stent pattern repeats itself over 3 mm and there are 8 struts around the circumferential, to put three cavities 40 per strut per section, the stent must be rotated three times per axial movement of 3 mm and the laser is fired 24 times.

As shown in FIG. 5, a sensor 80, such as a miniature video system, may be used to locate positions on the struts at which the cavities are to be placed. The locating can occur while the stent and balloon catheter or other medical device 10 is rotated. Once the desired position of the strut is located, the time at which a specific position of a strut that will spiral along the focal point of the excimer laser 70 can be calculated. Thus, after a fixed delay the excimer laser 70 can be activated to ablate a cavity 40 in the first coating layer 30. Given the high repetition rate of the excimer laser 70, the medical device 10 may continue to rotate while ablating when multiple shots of the laser are required to ablate fully to the surface 20 of the medical device 10 or partially to the surface 20 to form a deep cavity 40.

The method further comprises depositing a biologically active material 50 in at least a portion of the cavity 40. As discussed above, the biologically active material 50 is preferably deposited in the cavity 40 using a picoliter dispensing system. The picoliter dispensing system includes a first dispenser 90 that houses and dispenses the biologically active material 50. The first dispenser 90 is preferably located downstream the spiraling path of the stent or other medical device 10, as shown in FIG. 5. By knowing the timing of the ablation by the excimer laser 70 and the rotation and forward movement, the time that a particular cavity 40 will pass underneath the first dispenser 90 can be precisely calculated. Thus, after a fixed delay after the cavity 40 has been ablated by the excimer laser 70, the biologically active material 50 can be deposited into the cavity 40.

More than one biologically active material 50 may be used to fill one or more cavities 40. The different biologically active materials 50 may be deposited in the cavities 40 using different dispensers. Also, the biologically active material 50 can be distributed in the cavities 40 in any desired pattern.

The amount of biologically active material 50 deposited can be precisely calculated as the droplets can be counted individually and the size of each droplet can be precisely calculated by a laser scanning method. As the droplet size and the number of droplets can be precisely measured, the amount of biologically active material 50 injected into a cavity 40 can be calculated with a high degree of accuracy using the method of the present invention. In fact, about 100% of the biologically active material 50 that is dispensed is deposited into a cavity 40.

The present method further comprises forming a second coating layer 60 comprising a second polymer over the biologically active material 50 in the cavity 40. The second polymer may be the same as the first polymer. A second dispenser 100 with a second coating layer 60 composition can be placed downstream from the first dispenser 90 with the biologically active material 50.

The following example shows calculations for determining the number and size of cavities needed to deliver 20 μg of a biologically active material. In this example, the first coating layer 30 is about 20 micrometers thick, and comprises a biostable polymer, and the biologically active material 50 is paclitaxel.

Assuming that 1 μg of paclitaxel has a volume of 1 nanoliter to form a 20% solution or dispersion of paclitaxel, the 20,000 picoliters of paclitaxel can be combined with 80,000 picoliters of tetrahydrofuran (7HF) to form a 100,000 picoliter solution. If the drop size of the paclitaxel solution is selected to be 50 picoliters, 2000 droplets have to be applied to the cavities of the first coating layer so that the stent comprises the 20,000 picoliter or 20 μg of paclitaxel. The diameter of a 50 picoliter droplet is about 40 micrometer. Since the first coating layer has a thickness of about 20 micrometer, if the diameter of the cavities in the first coating layer is selected to be about 46 micrometer, one 50 picoliter droplet would fit in and fill a cavity. However, since the solution contains 20% paclitaxel after release of the solvent (THF), 20% of the cavity would be filled with paclitaxel. Dispensing two droplets in one cavity would thus result in a 40% filling of the cavity. A second dispenser with the polymer could be used to fill the cavities with a second coating layer.

Since 2000 droplets must be dispensed to apply the 20 μg of paclitaxel to the surface of the stent, and it is desirable to dispense two droplets of paclitaxel in each cavity, there must be 1000 different cavities along the stent surface. With a 9 mm stent having a design with four rings in a sinus shape with 9 repetitions of the curves along the circumference, there will be 14 cavities over one half curve. Since a half curve extends over 2250 micrometer (9 mm/4 rings), the cavities must be separated by 160 micrometer (2250 micrometer/14 cavities). In other words, the balloon must be rotated in a spiral with a pitch length of 160 micrometer. As known to one skilled in the art, similar calculations may be used for making other medical devices with other biologically active materials and coating layer materials.

The method of the present invention has many advantages including providing an efficient, cost-effective, and relatively safe manufacturing process for applying a biologically active material to a medical device. The present method allows for the biologically active material to be applied to the medical device as a final step in the manufacturing process such as after the stent has been crimped on a balloon catheter. Thus, this method minimizes the risk of loss of the biologically active material. In addition, the medical device can be packaged directly after carrying out the method of the present invention.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

1. A coated medical device comprising:
a medical device having a surface;
a first coating layer comprising a first polymer disposed on at least a portion of the surface;
at least one cavity in the first coating layer;
a first biologically active material deposited in the cavity; and
a second coating layer comprising a second polymer disposed over the biologically active material in the cavity.

2. The medical device of claim 1, wherein the second coating layer is substantially free of any biologically active material.

3. The medical device of claim 1, wherein the second coating layer is further disposed over the first coating layer.

4. The medical device of claim 1, wherein the biologically active material does not completely fill the cavity.

5. The medical device of claim 1, wherein the medical device is a stent.

6. The medical device of claim 1, wherein the first polymer is the same as the second polymer.

7. The medical device of claim 1, wherein the biologically active material is an anti-proliferative agent.

8. The medical device of claim 7, wherein the anti-proliferative agent is selected from the group consisting of paclitaxel, paclitaxel analogues, paclitaxel derivatives, and combinations thereof.

9. The medical device of claim 1, comprising a plurality of cavities in the first coating layer and wherein at least some of the cavities contain a second biologically active material.

10. The medical device of claim 1, wherein the cavities have different depths.

11. The medical device of claim 1, wherein the cavities have different shapes.

12. The medical device of claim 1, wherein the cavities are formed by laser ablation.

13. The medical device of claim 1, wherein the first biologically active material further comprises a polymer.

14. A stent comprising:
a sidewall comprising a plurality of struts each having a surface;
a first coating layer comprising a polymer disposed on at least a portion of the surface of at least one strut;
a plurality of cavities formed in the first coating layer by ablation with a laser;
a biologically active material deposited in the cavities; and
a second coating layer comprising a polymer disposed over the biologically active material in the cavities.

15. The stent of claim 14, wherein the biologically active material is an anti-proliferative agent selected from the group consisting of paclitaxel, paclitaxel analogues, paclitaxel derivatives, and combinations thereof.

16. A method of making a coated medical device having a surface wherein the method comprises:
forming on the surface a first coating layer comprising a first polymer;
laser ablating at least one cavity in the first coating layer;
depositing a biologically active material in the cavity; and
forming a second coating layer comprising a second polymer over the biologically active material in the cavity.

17. The method of claim 16, wherein the cavity is formed using an excimer laser or an ultrashort laser to ablate the first coating layer.

18. The method of claim 16, wherein the biologically active material is deposited in the cavity using a picoliter dispensing system.

19. The method of claim 16, wherein a plurality of cavities are ablated in the first coating layer and the cavities have different depths.

20. The method of claim 16, wherein the medical device is a stent comprising a plurality of struts and the surface is located on a strut.

21. The method of claim 16, wherein the first polymer is the same as the second polymer.

22. The method of claim 16, wherein the biologically active material is an anti-proliferative agent.

23. The method of claim 22, wherein the anti-proliferative agent is selected from the group consisting of paclitaxel, paclitaxel analogues, paclitaxel derivatives, and combinations thereof.

24. The method of claim 16, wherein the second coating layer is substantially free of any biologically active material.

25. The method of claim 16, wherein the second coating layer is further formed over the first coating layer.

26. The method of claim 16, wherein the biologically active material does not completely fill the cavity.

27. The method of claim 16, wherein the biologically active material further comprises a polymer.

28. A coated medical device comprising:
a medical device having a surface;
a first coating layer comprising a first polymer disposed on at least a portion of the surface;
at least one cavity in the first coating layer; and
a second coating layer comprising a second polymer and a biologically active material disposed over the cavity.
